(19) United States
(12) Patent Application Publication
(10) Pub. No.: US 2025/0069760 A1
(43) Pub. Date: Feb. 27, 2025

(54) System and Method for Correlating Diverse Medication Information

(71) Applicant: CoveredRx LLC, Palm Beach Gardens, FL (US)

(72) Inventor: Michael NAVIN, Palm Beach Gardens, FL (US)

(73) Assignee: CoveredRx LLC, Palm Beach Gardens, FL (US)

(21) Appl. No.: 18/454,218

(22) Filed: Aug. 23, 2023

(51) Int. Cl. G16H 70/40

(52) U.S. Cl. CPC G16H 70/40

(57) ABSTRACT

A system and method are presented for correlating diverse medication information from a plurality of medication search services where a system computer includes a storage having a table of information facilitating the normalization of data from a plurality of medication search services. The system facilitates simultaneous searching of a plurality of medication search services each comprising different medication information and normalizing of the diverse medication data to present a single view of all the diverse information for simultaneous comparison.

### BACKGROUND

### 1. Field of the Invention

The system relates to a system and method for providing prescription drug information to a user. More specifically, the system and method provide for a user to search and be presented with information related to numerous medications where the information is tailored to the user based on user information.

### 2. Description of Related Art

Medications and the ability to search for them via internet searching has been known for many years. Some companies have offered medication searching online where an individual may enter the name of a medication and the system will point the individual to multiple cash application websites, then to a pharmacy. The individual can then proceed to the pharmacy website to request filling of the prescription. The limitation of this system is that the individual cannot easily compare costs across sources.

In more recent years, medication search services such as SingleCare and Cost Plus have become available. These websites are connected to medication databases that include various medications with associated negotiated cash pricing. This allows a user to search the database for a medication and be provided with an associated cost. The user could then click on a link on the webpage that would provide them with instructions on how to redeem that cost. However, the individual does not have a truly accurate way to compare pricing from one source to another. For example, the prices of drugs are often variable depending on the source providing the drugs and on the insurance plan the user currently has.

These medication search services have their own databases and coverage. Searching one database (e.g., Cost Plus), does not give a user a full picture of the wider range of options and a more accurate representation of the market. Likewise, costs associated with a search are variable such that performing a search at a first time may result in a different price when performing that same search at a second time making it hard to truly do a cost comparison sequentially across a range of medication search services.

Still further, there is no ability for a user to search multiple medication search services databases and be provided with customized information based on user information where the system is Health Insurance Portability and Accountability Act of 1996 (HIPAA) compliant.

Accordingly, there is a need for a system and method that allows for searching over a broad range of medication search services to alleviate, and/or mitigate one or more of the aforementioned and other deleterious effects of prior art.

### SUMMARY

In one configuration, it is desired to provide a system and method that allows for simultaneous searching of a plurality of medication search service databases for a medication.

In another configuration, it is desired to provide a system and method that simultaneously searches a plurality of medication search service databases and provides pricing information to a user accounting for the user's personal information.

In still another configuration, it is desired to provide a system and method that simultaneously searches a plurality of medication search service databases accounting for the user's personal information and presents this information to a user and is HIPAA compliant.

A major technical problem with developing and implementing a system that allows for simultaneous searching of multiple different databases is that medication search services do not have consistency in the Nation Drug Code (NDC) used for medications. For example, the medication search services SingleCare and Cost Plus do not use consistent NDCs. This prevents cross-referencing of similar drugs listed in different databases. This then requires that a user sequentially search each database separately, which is time-consuming.

To address this problem, a system for searching different medication search services databases was developed with an external database that maps between various medication search services databases. This mapping can comprise a lookup table where a drug reference in a first medication search service database is normalized with a drug reference in a second medication search service database providing for simultaneous searching and for accurate data.

Accordingly, in one configuration, a system for searching and presenting medication information is provided that includes a master database of medication information corresponding to a first medication database and a second medication database where medication data for a first medication from the first medication database differs from medication data for the first medication from the second medication database.

Additionally, the system is provided such that a user may input their personal information, including insurance information, which can be used by the system to determine a user's adjusted insurance medication pricing to be presented to the user.

This new technology allows for users to shop for prescriptions and cost compare across various medication search services and providers. This provides users with information they wouldn't typically have access to in one easy-to-use technology that is HIPAA compliant.

In particular, the system provides for comprehensive results through aggregating multiple search services such that users are provided with a more comprehensive view of their prices for medications. Providing an interface to simultaneously search disparate databases and combining the results allows users to gain access to a wider range of options and provides a more accurate representation of the market.

The system further provides for increased cost savings because users can compare prices across multiple platforms simultaneously. Additionally, the convenience and timesaving of the system means that users will not have to individually query each database sequentially and separately. Users can conduct their searches in one place, compare prices, and access additional details without the need to switch between multiple applications or websites.

Another advantage is single access to diverse data sources that have partnerships with various pharmacies, insurance providers, or medication wholesalers. By aggregating these services, users can tap into a broader network of data sources, ensuring access to a more extensive and diverse range of pricing information.

For this application the following terms and definitions shall apply:

The term “data” as used herein means any indicia, signals, marks, symbols, domains, symbol sets, representations, and any other physical form or forms representing information, whether permanent or temporary, whether visible, audible, acoustic, electric, magnetic, electromagnetic or otherwise manifested. The term “data” as used to represent predetermined information in one physical form shall be deemed to encompass any and all representations of the same predetermined information in a different physical form or forms.

The term “network” as used herein includes both networks and internetworks of all kinds, including the Internet, and is not limited to any particular type of network or inter-network.

The terms “first” and “second” are used to distinguish one element, set, data, object or thing from another, and are not used to designate relative position or arrangement in time.

The terms “coupled”, “coupled to”, “coupled with”, “connected”, “connected to”, and “connected with” as used herein each mean a relationship between or among two or more devices, apparatus, files, programs, applications, media, components, networks, systems, subsystems, and/or means, constituting any one or more of (a) a connection, whether direct or through one or more other devices, apparatus, files, programs, applications, media, components, networks, systems, subsystems, or means, (b) a communications relationship, whether direct or through one or more other devices, apparatus, files, programs, applications, media, components, networks, systems, subsystems, or means, and/or (c) a functional relationship in which the operation of any one or more devices, apparatus, files, programs, applications, media, components, networks, systems, subsystems, or means depends, in whole or in part, on the operation of any one or more others thereof.

The terms “process” and “processing” as used herein each mean an action or a series of actions including, for example, but not limited to, the continuous or non-continuous, synchronous or asynchronous, routing of data, modification of data, formatting and/or conversion of data, tagging or annotation of data, measurement, comparison and/or review of data, and may or may not comprise a program.

In one configuration, a system for correlating diverse medication information from a plurality of medication search services is provided comprising, a system computer accessible by a user computer via a network connection, and a storage accessible by the system computer including a table of data that includes medication information selected from the group consisting of: medication name, medication dosage, medication code data, or combinations thereof. The system is provided such that when the system computer receives a medication search query from a user computer, the system computer cross-references the medication search query with first and second medication search service data from the table, and the system computer transmits the first and second medication search service data to a first medication search service computer and a second medication search service computer respectively. The system is further provided such that the first and second medication search service computers provide first and second medication information respectively to the system computer in response to the first and second medication search service data, and the system computer normalizes the first and second medication information with the table of data and presents a list of prices corresponding to the medication search query.

In another configuration, a method for correlating diverse medication information from a plurality of medication search services with a system computer connected to a network and having a storage, the method comprising the steps of: the system computer receiving from a user computer a medication search query, and the system computer searching the storage that comprises a table of data that includes medication information selected from the group consisting of: medication name, medication dosage, medication code data, or combinations thereof. The method further comprises the steps of: the system computer cross-referencing the medication search query with first and second medication search service data from the table, and the system computer simultaneously transmitting the first and second medication search service data to a first medication search service computer and a second medication search service computer respectively. Finally, the method comprises the steps of: the system computer receiving first and second medication information from the first and second medication search service computers respectively, and the system computer normalizing the first and second medication information with the table of data and presenting a list of prices corresponding to the medication search query

The above-described and other features and advantages of the present disclosure will be appreciated and understood by those skilled in the art from the following detailed description, drawings, and appended claims.

### DESCRIPTION OF THE DRAWINGS

FIG. 1 is a block diagram showing a system for correlating diverse medication information from various medication search service databases in accordance with embodiments of the invention;

FIGS. 2A-2C are flow diagrams of the system process according to FIG. 1; and

FIGS. 3-13 are various screen shots of one configuration of the system according to FIG. 1.

### DETAILED DESCRIPTION

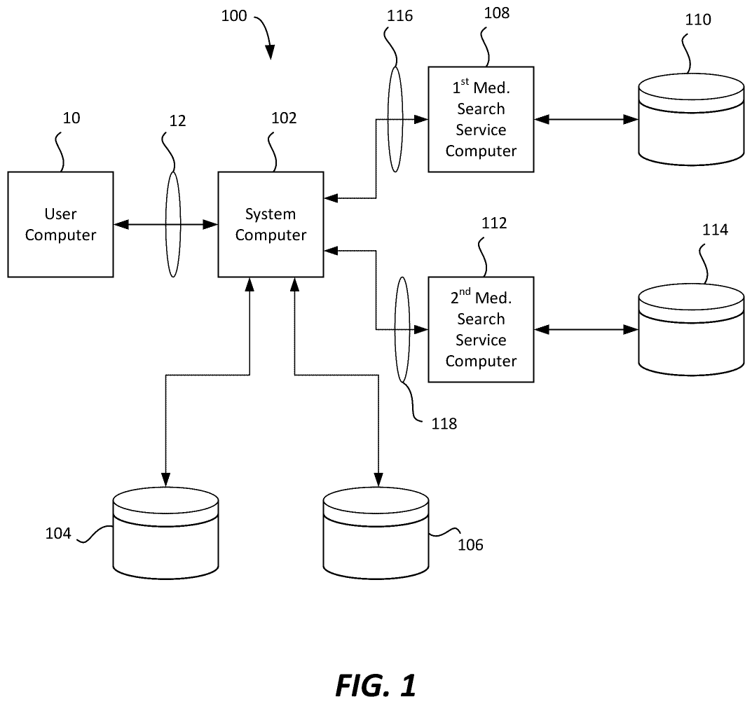

Referring to the drawings and to FIG. 1, a system for searching medication search service databases 100 is provided. The system comprises a computer 102 that is accessible by a user computer 10 via a network connection 12. Computer 102 has a storage(s) 104, 106 that may comprise one or more databases. In one configuration, storage 104 may be used to hold user information. This could comprise, for example, a name, date of birth, medical history data, medication data, insurance information, physician information and so on. It is contemplated that computer 102 and storage 104 will be maintained compliant with HIPAA regulations. Storage 106 may comprise a database of information for cross-referencing other databases information.

Also illustrated in FIG. 1 is computer 108, which is connected to computer 102 via a network connection 116. Computer 108 has a storage 110 accessible thereto and which may comprise a database. Also shown is computer 112, which is connected to computer 102 via a network connection 118. Computer 112 has a storage 114 accessible thereto and which may comprise a database.

Computers 108, 112 may comprise medication search services. These may comprise a first and a second medication service such as SingleCare and Cost Plus that allow a user to search for medications. In one example, the medication search service may provide a cost for a searched medication and provide a pharmacy that can fulfill this medication request. In this example, the user is able to access the system for searching medication search service databases as a single portal when connecting to computer 102. The user can enter a medication name and dosage into computer 102, which in turn, will then search for this medication among a plurality of medication search service databases.

The system 100 faces the challenge that the NDC that is used for identifying a medication associated with the first medication search service on computer 108 and saved in storage 110, may be different from the NDC used to identify that same medication by a second medication search service via computer 112 and saved on storage 114.

To address this problem, a relational database, which could take the form of a look up table, is generated by system 100 and saved on storage 106 for access by computer 102. This allows system 100 to search other databases by the user entering a medicine name into user computer 10 that is then transferred to computer 102. Computer 102 will then take the medication description and cross reference it with a table of medication information such that the computer 102 will then take that cross-referenced information from the look up table and send that to the first and second medication search service computers 108, 112. As such, the database of medication data in storage 106 is normalized so that comparable data from both the first and second medication search service computers 108, 112 can be simultaneously searched and presented to the user allowing for the computer 102 to aggregate various prescription cost options in one location.

The aggregation of multiple medication search services into a single application allows users to search for prices across different platforms using different medication description data simultaneously.

Additionally, further functionality includes cross-referencing with the user's insurance information stored in storage 104. Storage 104 can include a Profiles Database that encompasses user profiles and preferences. This database maintains information like user authentication data, insurance details, and personalized settings.

Computer 102 can utilize the user information to cross-reference prices of medications with their own insurance coverage. This obviously enables users to in one search, identify the most cost-effective options including their specific insurance plans. The user's insurance information will be pulled from various integrations with either Third-Party Administrators (TPAs), Prescribed minimum Benefits (PBMs), and other partnership sources.

Other features of system 100 include the ability to provide customized user experience. For example, the system 100 may, in one configuration, provide personalized features such as user profiles, medication reminders, prescription history tracking, or medication substitution suggestions. These features enhance the user experience and provide added value beyond basic search functionality while at the same time, allowing for simultaneous searching across a variety of different medication search services.

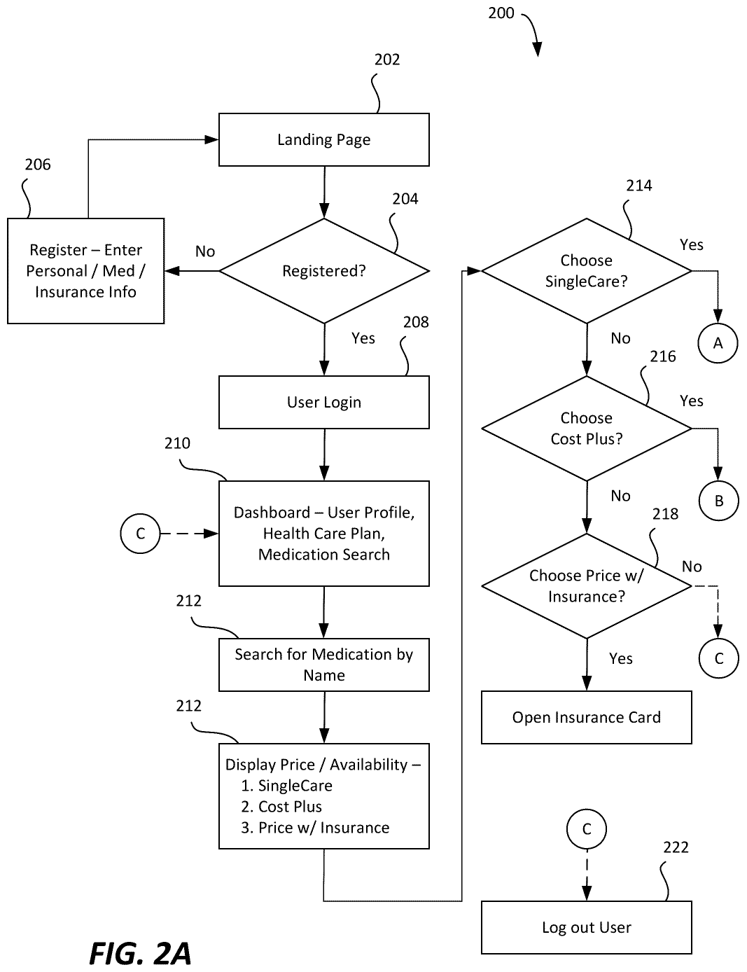

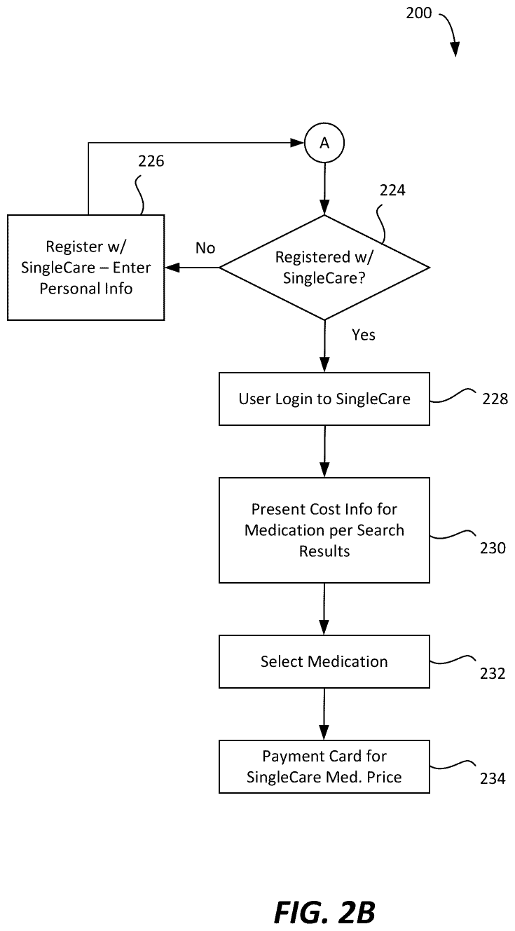

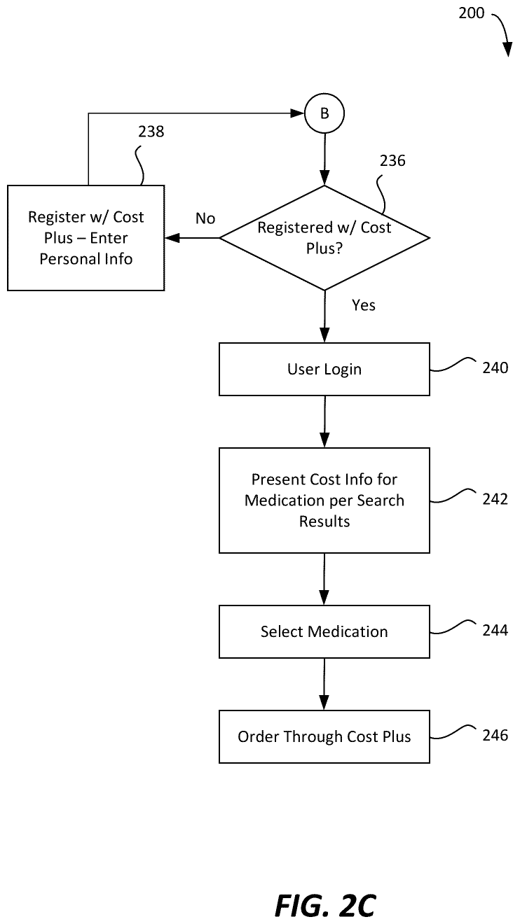

Referring now to FIGS. 2A-2C, a flow chart is provided that illustrates the process flow 200 according to system 100.

Initially the user can access landing page 202 on computer 102 via the user computer 10. Once on landing page 202, the user is presented with the option to register 204 with system 100. If the user has not previously registered, the user can enter their registration information 206 into system 100, which will be saved on storage 104. This information can include the user's personal information, their medical information/history and insurance information. Once the user has registered, they user can then Login 208 to computer 102.

Once the user has logged into the system 100, a dashboard 210 is presented to the user. The dashboard may include a presentation of a User Profile, it may include the user's Health Care Plan, and may further provide a search window for searching a medication(s).

At this point, the user has the option to search for a medication(s) 212. This may simply take the form of a search window where the user can enter a common name for the medication along with a dosage, a quantity and form. The system can be provided with a relational database as previously described that relates various names of a medication (e.g., generic names, brand names, common descriptors, etc.) to search for various medications.

Once the medication is identified, the medication may be displayed 214 to the user. In the example provided in FIG. 2A, the system may display results from SingleCare, Cost Plus and the price if purchased through the user's insurance.

Once the search results are returned, the user has the option to select from among the results. In this example, the user has the option to select the SingleCare results 214. If the user decides to select the SingleCare results, the process continues to FIG. 2B. If not, the user has the option to select Cost Plus results 216. If the user decides to select the Cost Plus results, the process continues to FIG. 2C. If not, the user has the option to select the price connected with their own insurance coverage 218. If the user decides to select the price connected with their own insurance, the system can proceed to open their insurance card 220. If not, the system may simply wait for the user and in the event no option is selected, the system may automatically time out and log the user off 222, or the system may revert back to the dashboard. These options are displayed in dashed line to illustrate that multiple options are available.

Referring to FIG. 2B, if the SingleCare option was selected the user is presented with an option to login or register with SingleCare 224. If the user has not previously registered with SingleCare, the user then can register 226 providing personal information and any other required information including, for example, payment information. If the user has previously registered, the user may then login to the SingleCare portal 228. The user will then be presented with cost information for the medication that was previously searched 230. The user can then select the medication 232 for purchase and then the system proceeds to payment card for SingleCare Medication price 234.

Referring to FIG. 2C, if the Cost Plus option was selected the user is presented with an option to login or register with Cost Plus 236. If the user has not previously registered with Cost Plus, the user then can register 238 providing personal information including payment and/or other information such as, for example, physician information. If the user has previously registered, the user may then login to the Cost Plus portal 240. The user will then be presented with cost information for the medication that was previously searched 242. The user can then select the medication 244 for purchase and then the system proceeds to follow instructions for Cost Plus Medication price 246.

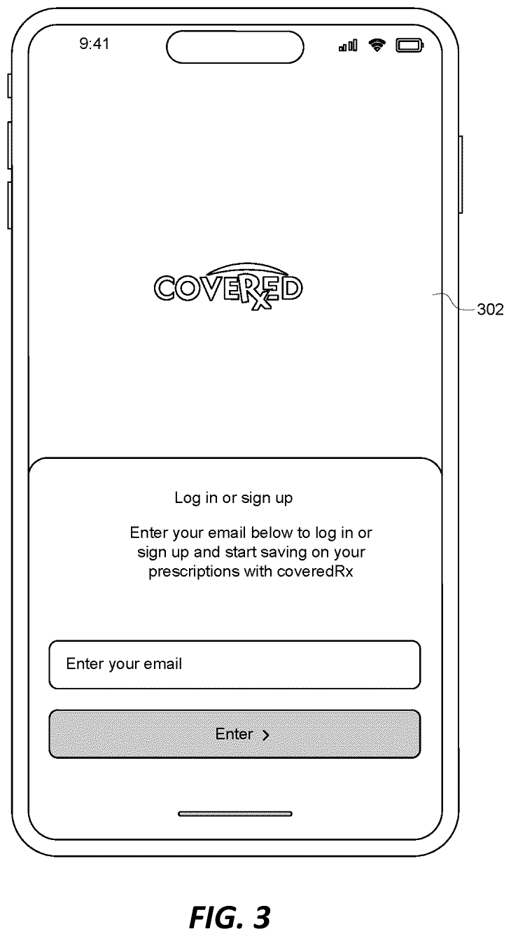

Referring now to FIGS. 3-13, screen shots of the system and processes described in connection with FIGS. 1-2C are illustrated. This application is shown as a mobile device app, however, it is contemplated that the system may be usable on any platform with internet connectivity.

FIG. 3 is an illustration of a landing page 302 where a user can login or register.

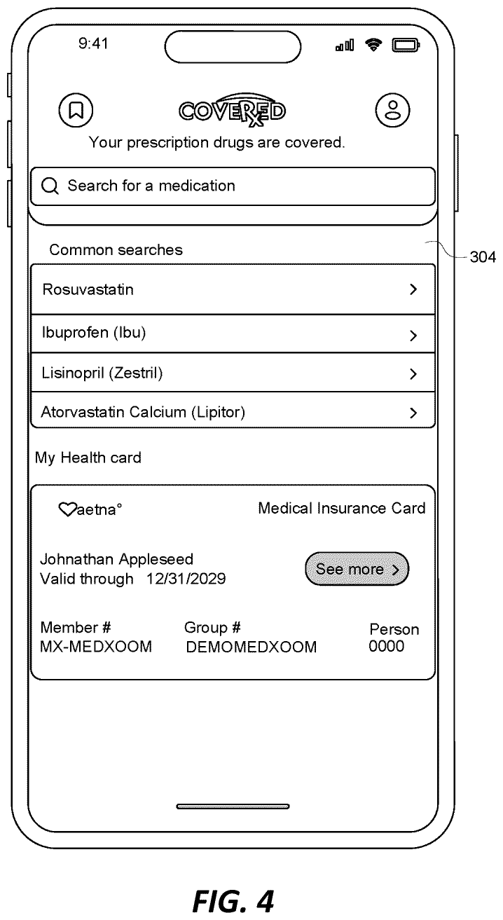

FIG. 4 is a screen shot where a user may search for a medication 304. In the example shown, common medication searches may be automatically populated onto the search page. In one configuration, the system will remember previous searches the user performed and can populate the search page automatically with previous medications that were searched. Also shown is the user's medical insurance information and the user's name.

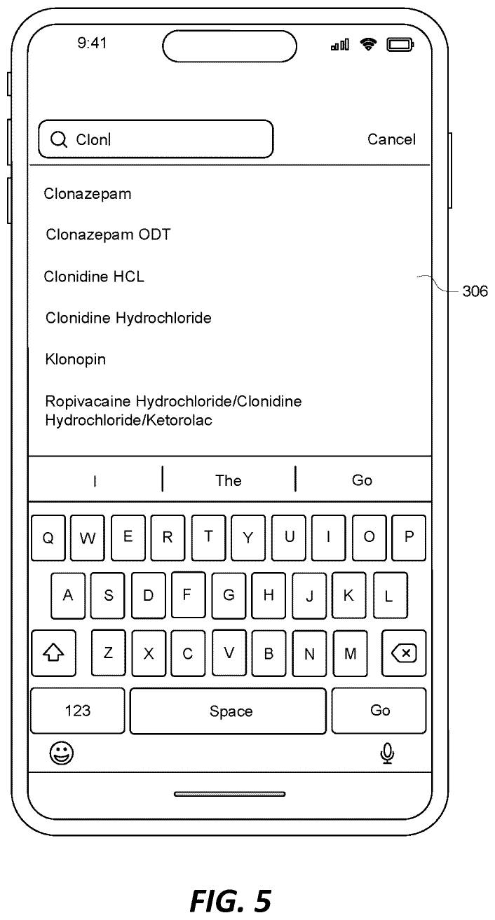

FIG. 5 is a screen shot of the search window according to FIG. 4, however, the user has begun to enter a medication name into the search window. As the user progressively enters the medication name, various medications that match with the information provided thus far will be displayed adjacent to the window. In this example, the user has entered “Clon” and in response the system is displaying “Clonazepam”, “Clonazepam ODT”, “Clonidine HCL”, “Clonidine Hydrochloride” and “Klonopin”, “Ropivacaine Hydrochloride/Clonidine Hydrochloride/Ketorolac” as possible matches enabling the user to select the medication that may match the partially spelled medication.

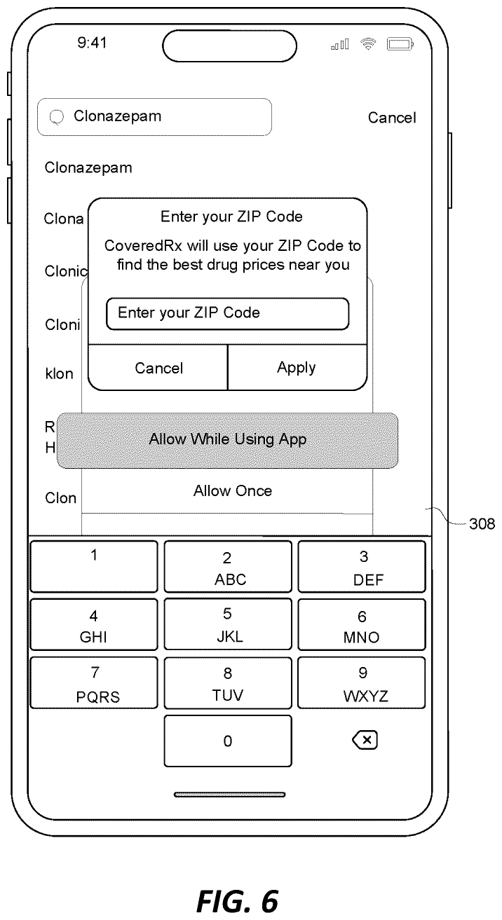

Referring to FIG. 6, once the user has selected or entered the name of the medication they want, the system will then prompt the user to enter a ZIP Code 308, which will be used to find the medication prices near the user's ZIP Code. It is contemplated that once the user enters their personal information into the system, the system can remember this information and automatically use it to provide the pricing information. Alternatively, the user may be away from their primary residence and be residing at a secondary residence. The system could prompt the user to select a saved location or allow the user to enter a manual location (e.g., if the user is on vacation).

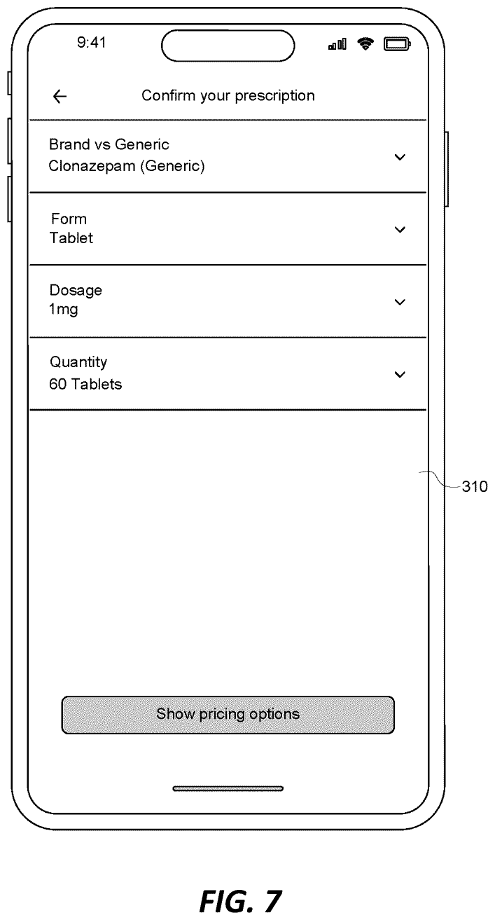

Referring to FIG. 7, once the user has selected or entered their ZIP Code information, the system will then provide a listing of the selected medication including the form (e.g., tablet), the dosage (e.g., 1 mg) and the quantity of the prescription (e.g., 60 tablets) 310. If this is correct, the user can then select the Show pricing options tab.

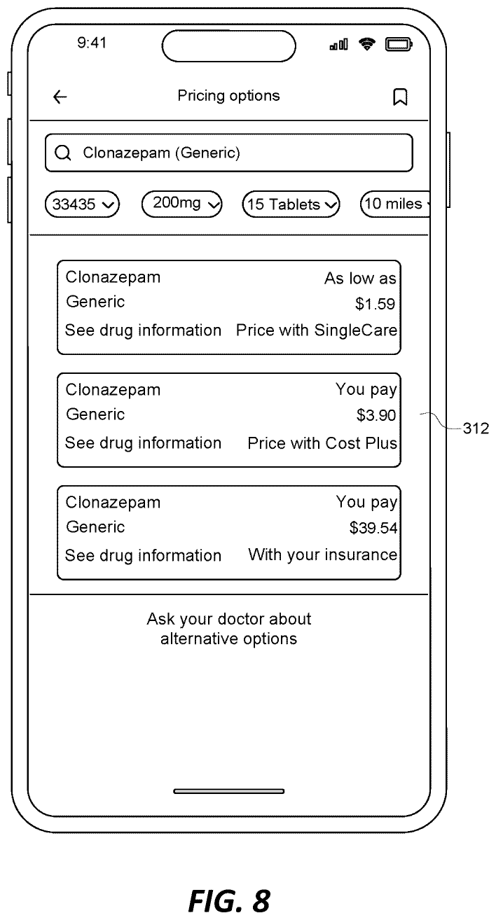

Once the user has selected the Show pricing options tab as per FIG. 7, the system advances to FIG. 8 showing various prices for the selected medication 312. In this instance, a price for SingleCare is illustrated, a price for Cost Plus is shown, and a cost associated with a generic version of the medication with the user's insurance is shown.

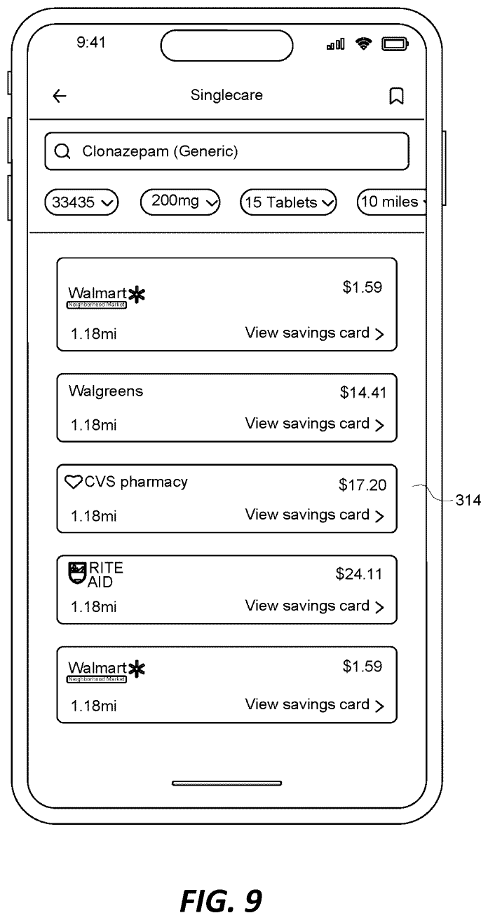

Referring to FIG. 9, the user has selected the SingleCare option and is then presented with various pricing for the searched medication from various locations 314. The system may, in one configuration, provide proximity of a pharmacy to the user where the medication can be obtained. This information can be based on the location information previously provided.

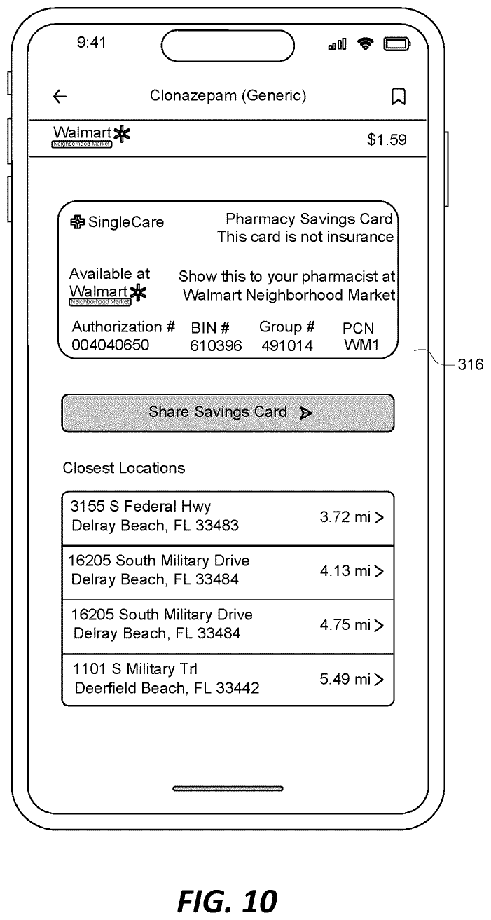

Referring now to FIG. 10, the user has selected a particular pharmacy where the search medication can be purchased 316. On this page, the user is provided with the name of the pharmacy (e.g., Walmart) along with a price. The user can use their SingleCare Pharmacy Savings Card if they have one. Various information about the medication may also be shown including the Authorization #, the BIN #, the Group # and the PCN. The closet location(s) for the pharmacy can be presented to the user as illustrated. At this point, the user has the option to select the Share Savings Card tab.

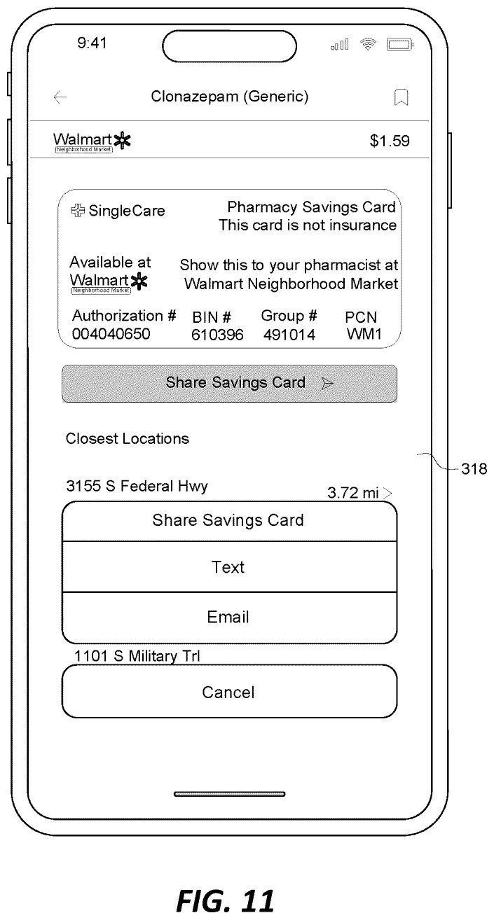

Once the user has selected the Share Saving Card tab, the system proceeds to FIG. 11 and may prompt the user as to the method of sharing such as, Text or Email 318.

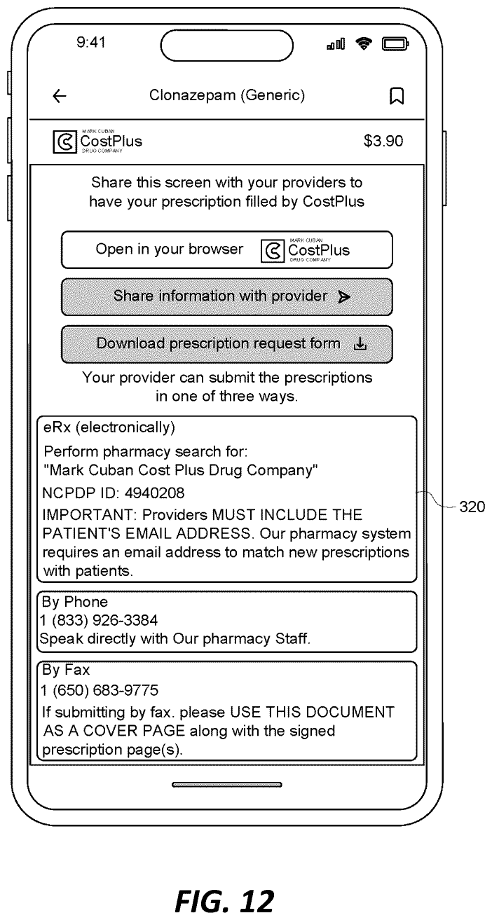

Referring to FIG. 12, the system proceeds to Share this screen with your providers to have your prescription filled 320. In the example provided in FIG. 12, the user has selected the Cost Plus option from FIG. 8.

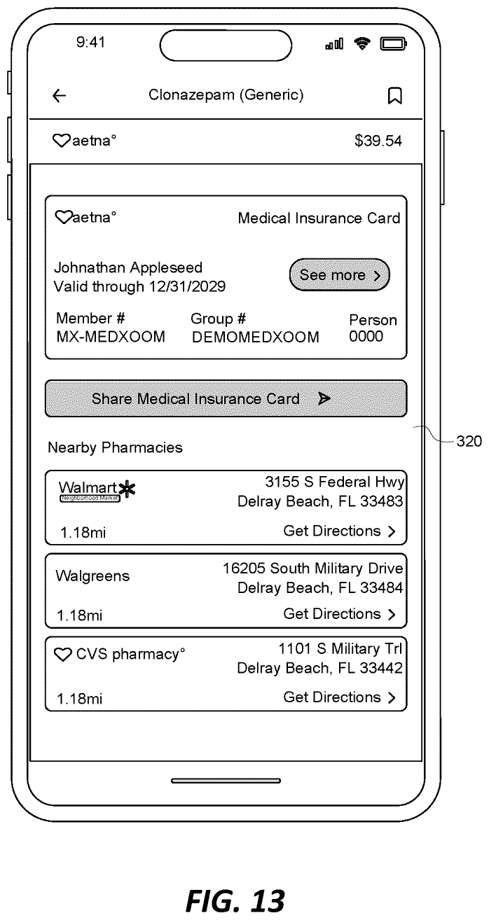

Finally, in FIG. 13 the user has the option to share their medical insurance card 320 if that is the preferred option selected from FIG. 8.

It should be noted that, while various functions and methods have been described and presented in a sequence of steps, the sequence has been provided merely as an illustration of one advantageous embodiment, and that it is not necessary to perform these functions in the specific order illustrated. It is further contemplated that any of these steps may be moved and/or combined relative to any of the other steps. In addition, it is still further contemplated that it may be advantageous, depending upon the application, to utilize all or any portion of the functions described herein.

Additionally, while the present disclosure has been described with reference to one or more exemplary embodiments, it will be understood by those skilled in the art that various changes may be made, and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure is not limited to the particular embodiment(s) disclosed as the best mode contemplated, but that the disclosure will include all embodiments falling within the scope of the appended claims.

1. A system for correlating diverse medication information from a plurality of medication search services comprising:
a system computer accessible by a user computer via a network connection;
a storage accessible by the system computer including a table of data that includes medication information selected from the group consisting of: medication name, medication dosage, medication code data, or combinations thereof;
said storage further includes user information selected from the group consisting of name, zip code or combinations thereof
wherein said system computer comprises software adapted to receive a medication search query from a user computer related to a medication, said system computer cross-references the medication search query with first and second medication search service data from the table;
wherein the first medication search service data includes a first medication code and the second medication search service data includes a second medication code, where the first medication code is different than the second medication code;
wherein said system computer simultaneously transmits the first and second medication search service data to a first medication search service computer and a second medication search service computer respectively;
wherein the first and second medication search service computers provide first and second medication information respectively to said system computer in response to the first and second medication search service data;
wherein said system computer normalizes the first and second medication information with the table of data and presents a list of prices corresponding to the medication search query.

2. The system of claim 1, wherein the user information is further selected from the group consisting of: date of birth, address, medical information or history, insurance information, user preferences or combinations thereof.

3. The system of claim 1, wherein said software comprises a graphic user interface (GUI) adapted to receive location information.

4. The system of claim 3, wherein the system presents pricing information based in part on the location information.

5. The system of claim 1, wherein said software comprises a graphic user interface (GUI) adapted to receive the medication information associated with the medication search query.

6. The system of claim 5, wherein the GUI comprises a window adapted to receive text.

7. The system of claim 6, wherein when the medication search query is entered, the system will dynamically search the database of medications and provide medication options that can be selected based on the entered data.

8. The system of claim 1, wherein the list of prices corresponding to the medication search query further includes pharmacy information selected from the group consisting of: pharmacy name, pharmacy location or distance.

9. A method for correlating diverse medication information from a plurality of medication search services with a system computer connected to a network and having a storage, the method comprising the steps of:
the system computer receiving from a user computer a medication search query;
the system computer searching the storage that comprises a table of data that includes medication information selected from the group consisting of: medication name, medication dosage, medication code data, or combinations thereof, and user information selected from the group consisting of: name, zip code or combinations thereof;
the system computer cross-referencing the medication search query with first and second medication search service data from the table;
wherein the first medication search service data includes a first medication code and the second medication search service data includes a second medication code, where the fist medication code is different than the second medication code;
the system computer simultaneously transmitting the first and second medication search service data to a first medication search service computer and a second medication search service computer respectively;
the system computer receiving first and second medication information from the first and second medication search service computers respectively; and
the system computer normalizing the first and second medication information with the table of data and presenting a list of prices corresponding to the medication search query.

10. The system of claim 9, wherein the user information is further selected from the group consisting of: date of birth, address, medical information or history, insurance information, user preferences or combinations thereof.

11. The method of claim 9, further comprising the step of the system computer receiving location information associated with the user.

12. The method of claim 11, wherein the list of prices is based in part on the location information.

13. The method of claim 9, wherein the system computer includes software adapted to receive a medication description for the medication search query.

14. The method of claim 13, wherein the software comprises a window adapted to receive text.

15. The method of claim 14, wherein when the text is being entered, the system dynamically searches the table of data and provides medication options based on the entered data.

16. The method of claim 15, wherein the medication options are adapted to be selected by the user.

17. The method of claim 9, wherein the list of prices corresponding to the medication search query further includes pharmacy information selected from the group consisting of: pharmacy name, pharmacy location or distance.

18. The system of claim 1,
wherein the first medication information provided by the first medication search service computer includes a listing of where the medication can be purchased from a first group of retail outlets compiled by the first medication search service computer and includes a cost for the medication offered by each of the retail outlets in the first group; and
wherein the second medication information provided by the second medication search service computer includes a listing of where the medication can be purchased from a second group of retail outlets compiled by the second medication search service computer and includes a cost for the medication offered by each of the retail outlets in the second group.

19. The method of claim 9, further comprising the steps of:
providing the first medication information with the first medication search service computer, the first medication information including a listing of where the medication can be purchased from a first group of retail outlets compiled by the first medication search service computer and includes a cost for the medication offered by each of the retail outlets in the first group; and
providing the second medication information with the second medication search service computer, the second medication information including a listing of where the medication can be purchased from a second group of retail outlets compiled by the second medication search service computer and includes a cost for the medication offered by each of the retail outlets in the second group.
